(19) United States
(12) Patent Application Publication
(10) Pub. No.: US 2014/0222013 A1
(43) Pub. Date: Aug. 7, 2014

(54) INTRA-OCULAR LENS INJECTION CARTRIDGE

(71) Applicants: Sanjay Ram Swaroop Argal, Vadodara (IN); Munavvar Tahir Hussain, Vadodara (IN)

(72) Inventors: Sanjay Ram Swaroop Argal, Vadodara (IN); Munavvar Tahir Hussain, Vadodara (IN)

(73) Assignee: Jagrat Natavar DAVE, Vadodara (IN)

(21) Appl. No.: 14/238,250

(22) PCT Filed: Aug. 7, 2012
(86) PCT No.: PCT/IB2012/054025

§ 371 Date: Feb. 11, 2014

(30) Foreign Application Priority Data
Aug. 11, 2011 (IN) 2265/MUM/2011

(51) Int. Cl. A61F 2/16

(52) U.S. Cl. CPC A61F 2/1678; A61F 2002/1686
USPC 606/107; 264/328.1

(57) ABSTRACT

A cartridge for implantation of a deformable intraocular lens (IOL) through a small incision in the eye, including a body portion including a first section including a first lens delivery passageway configured to contain an IOL readied for implantation, and a second section including a second lens delivery passageway connected to the first passageway, configured to fold the IOL when the IOL is pushed through the second passageway, and a nozzle portion extending from the body portion, the nozzle portion including a third passageway and a tip for insertion through the incision in the eye, the second passageway extending to the third passageway, the third passageway configured to transfer the folded IOL into an incision in an eye when the folded IOL is pushed through the nozzle. Related apparatus and methods are also described.

### RELATED APPLICATION

This application claims the benefit of priority of Indian Patent Application No. 2265/MUM/2011 filed Aug. 11, 2011, the contents of which are incorporated herein by reference in their entirety.

### FIELD AND BACKGROUND OF THE INVENTION

The present invention, in some embodiments thereof, relates to a cartridge for injection of an intraocular Lens (IOL), and more particularly, but not exclusively, to a cartridge sized and shaped for a specific IOL.

A cataract is a clouding that develops in a crystalline lens of an eye or in its envelope, varying in degree from slight to complete opacity and obstructing passage of light. Early in development of an age-related cataract, the power of the lens may be increased, causing near-sightedness (myopia), and a gradual yellowing/opacification of the lens may reduce the perception of blue colors. Cataracts typically progress slowly, causing vision loss, and are potentially blinding if untreated. Cataracts usually affect both eyes, but almost always one eye is affected earlier than the other.

When a cataract is sufficiently developed to be removed by surgery, the most effective and common treatment is to make an incision (capsulotomy) into the capsule of the cloudy lens in order to surgically remove the lens. There are two types of eye surgery which are commonly used to remove cataracts: extra-capsular (extra-capsular cataract extraction, or ECCE) and intra-capsular (intra-capsular cataract extraction or ICCE), typically followed by an implant of a PMMA (polymethylemethacrylate) lens.

Extra-capsular (ECCE) surgery consists of removing the lens but leaving the majority of the lens capsule intact. High frequency sound waves are sometimes used to break up the lens before extraction (phacoemulsification).

Intra-capsular (ICCE) surgery involves removing an entire lens from an eye, including the lens capsule, but it is rarely performed in modern practice.

In either extra-capsular surgery or intra-capsular surgery, a cataractous lens is removed and replaced with a plastic lens (an intraocular lens implant) which stays in the eye permanently.

An intraocular lens (IOL) is an implanted lens in the eye, usually replacing the existing crystalline lens because it has been clouded over by a cataract, or as a form of refractive surgery to change the eye's optical power. It usually consists of a small plastic lens with plastic side struts, called haptics, to hold the lens in place within the capsular bag inside the eye.

Cataract operations are usually performed using a local anesthetic and a patient is typically allowed to go home the same day. Recent improvements in intraocular technology now allow cataract patients to choose soft foldable lenses made of silicon & acrylics, which have become more popular because of the ability to fold or roll the lens, and these lenses can be inserted through a smaller incision.

U.S. Pat. No. 4,681,102 to Bartell discloses an apparatus and method for inserting an intra-ocular lens into an eye. The apparatus permits the insertion of the intra-ocular lens through small incisions, of the order of several millimeters, such as are employed in the phacoemulsification technique of cataract removal. The intra-ocular lens is placed within a hinged, generally cylindrical load chamber, having a pair of flanges. The load chamber is folded around the intra-ocular lens, so that the lens itself becomes folded or rolled along its length. The load chamber is fitted into an injector portion, which has a slot which is keyed to the flanges of the load chamber. The injector portion and load chamber are then fitted into an insertion cone which defines a lumen for passage of the intra-ocular lens through the lumen. A plunger inserted into the injector portion is used to push the intra-ocular lens through the lumen and into the eye. The folded intraocular lens gradually flowers out as it emerges from the distal end of the insertion cone.

U.S. Pat. No. 5,275,604 to Rheinish et al describes an apparatus and associated method for deforming and inserting a flexible intraocular lens into an eye which permits the size of an ocular incision to be significantly smaller than the diameter of the intraocular lens being implanted, and its embodiments are suitable for use with lenses having radial flange or projecting filament haptics. The apparatus includes a contoured duct with a pair of internal guiding grooves configured to engage peripheral edges of the lens, curling the lens as it is advanced along the longitudinal duct axis from the elongated inlet to the coaxially aligned generally circular outlet of the duct. The guiding grooves are mutually opposed and converge along the length of the duct to essentially the periphery of the duct outlet. A lens is implanted utilizing this apparatus by loading the lens into a receiving chamber adjacent to the lens-curling contoured duct, inserting the outlet or a cannular probe communicating coaxially with said outlet into the ocular incision, and advancing the lens through the duct and then through the outlet into the eye.

Additional background art includes:

U.S. Pat. No. 4,681,102 to Bartell;

U.S. Pat. No. 5,494,484 to Feingold;

U.S. Pat. No. 5,499,987 to Feingold;

U.S. Pat. No. 5,616,148 to Eagles et al;

U.S. Pat. No. 5,653,715 to Reich et al;

U.S. Pat. No. 4,573,998 to Mazzocco; and

U.S. Pat. No. 5,810,834 to Heyman.

### SUMMARY OF THE INVENTION

According to an aspect of some embodiments of the present invention there is provided a cartridge for implantation of a deformable intraocular lens (IOL) through a small incision in the eye, including a body portion including a first section including a first lens delivery passageway configured to contain an IOL readied for implantation, and a second section including a second lens delivery passageway connected to the first passageway, configured to fold the IOL when the IOL is pushed through the second passageway, and a nozzle portion extending from the body portion, the nozzle portion including a third passageway and a tip for insertion through the incision in the eye, the second passageway extending to the third passageway, the third passageway configured to transfer the folded IOL into an incision in an eye when the folded IOL is pushed through the nozzle.

According to some embodiments of the invention, the first lens delivery passageway is configured to contain an IOL in which the lens part is not folded.

According to some embodiments of the invention, the second lens delivery passageway includes passageway walls which progressively narrow the second passageway, the narrowing of the walls being symmetrical around a long axis connecting the first, second, and third passageway.

According to some embodiments of the invention, passageway walls progressively narrow at an angle designed for minimizing stress on the IOL when it folds. According to some embodiments of the invention, passageway walls progressively narrow at angles which change along the passageways. According to some embodiments of the invention, passageway walls progressively narrow at an angle configured for minimizing stress on the IOL based, at least in part, on a diameter of the IOL. According to some embodiments of the invention, passageway walls progressively narrow at an angle configured for minimizing stress on the IOL based, at least in part, on a diopter of the IOL.

According to some embodiments of the invention, having studs for connection to an IOL injector which includes a plunger for pushing the IOL along the lens delivery passageways.

According to some embodiments of the invention, having a shape for mating to an IOL injector which includes a plunger for pushing the IOL along the lens delivery passageways.

According to an aspect of some embodiments of the present invention there is provided a kit for implantation of a deformable intraocular lens (IOL) through a small incision in the eye, including the cartridge of any one of the above embodiments, and an unfolded intraocular lens.

According to some embodiments of the invention, the unfolded intraocular lens is stored within the first lens delivery passageway.

According to some embodiments of the invention, further including a slippery agent for reducing friction between the IOL and the cartridge.

According to an aspect of some embodiments of the present invention there is provided a method for manufacture of a cartridge for implantation of a deformable intraocular lens (IOL) through a small incision in the eye, (a) the cartridge including a body portion including a first section including a first lens delivery passageway configured to contain an unfolded IOL readied for implantation, and a second section including a second lens delivery passageway connected to the first passageway, configured to fold the IOL when the IOL is pushed through the second passageway, and a nozzle portion connected to the body portion, the nozzle portion including a third passageway and a tip for insertion through the incision in the eye, the second passageway extending to the third passageway, the third passageway configured to transfer the folded IOL into an incision in an eye when the folded IOL is pushed through the nozzle, and (b) the manufacturing method including forming the cartridge by molding material to produce the cartridge.

According to some embodiments of the invention, the molding includes injection molding. According to some embodiments of the invention, the material includes a plastic. According to some embodiments of the invention, the material includes one of the group consisting of polypropylene, and low-density polyethylene.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a simplified illustration of an example intraocular lens (IOL), showing a lens and attached haptics;

FIGS. 1B-1D are simplified illustrations of examples of different IOL designs;

FIGS. 1E and 1F are simplified illustrations of an example embodiment of an IOL injection cartridge, functionally connected to an IOL injector;

FIGS. 2A-2H are simplified illustrations of an example embodiment of an IOL injection cartridge, constructed according to an example embodiment of the invention, and of an IOL advancing along passageways in the cartridge;

FIGS. 3A-3C are additional simplified illustrations of the example embodiment of the cartridge of FIGS. 2A-2H;

FIGS. 4A-4E are additional simplified illustrations of the example embodiment of the cartridge of FIGS. 2A-2H;

FIGS. 5A-5D are additional simplified illustrations of the example embodiment of the cartridge of FIGS. 2A-2H;

FIGS. 6A-6C are additional simplified illustrations of the example embodiment of the cartridge of FIGS. 2A-2H; and

FIGS. 7A and 7B are additional simplified illustrations of the example embodiment of the cartridge of FIGS. 2A-2H.

### DESCRIPTION OF SPECIFIC EMBODIMENTS OF THE INVENTION

The present invention, in some embodiments thereof, relates to a cartridge for injection of an Intra-Ocular Lens (IOL), and more particularly, but not exclusively, to a cartridge sized and shaped for a specific IOL.

Rheinish et al describe a practitioner loading an intraocular lens (IOL) into a receiving chamber adjacent to a lens-curling, also termed lens-folding, contoured duct and advancing the IOL through the duct and then through an outlet into the eye.

The present invention, in some embodiments thereof, provides a cartridge pre-loaded with an IOL stored in the cartridge. The pre-loading enables a loading to be performed in a controlled environment, where an IOL is packaged in an IOL injection cartridge embodiment of the present invention. The packaging of the IOL can be inspected, and determined to be correct, all under no time constraints imposed by performing the loading whilst operating on a patient.

The present invention, in some embodiments thereof, reduces stress on an IOL while the IOL is being folded for insertion into an eye.

The present invention, in some embodiments thereof, provides an optimal lens-curling surface for the IOL size and the IOL power selected by the practitioner.

The present invention, in some embodiments thereof, provides a pre-packaged IOL and cartridge which are optionally mated to provide an optimal lens-curling surface for the IOL packaged inside.

The present invention, in some embodiments thereof, provides an IOL injection cartridge with a symmetric, gradually narrowing bore. The symmetric bore effects a folding of the lens at edges of the lens, reduces the amount of stress, and/or energy, and/or pressure, imparted to the lens by folding, and avoids damage to the lens. The gradual narrowing of the bore effects a gradual folding of the lens, and also positions, optionally folds, haptics attached to the lens as it passes down the bore, avoiding damage to the haptics.

The present invention, in some embodiments thereof, provides a cartridge which effects symmetric folding, with minimal risk to damage lens haptics.

The present invention, in some embodiments thereof, provides a cartridge which effects symmetric folding, with a rotational symmetry with respect to a center of a bore, or passageway, in the cartridge.

The present invention, in some embodiments thereof, provides a cartridge which has a gradually narrowing bore with a round-cornered rectangular cross section.

The present invention, in some embodiments thereof, provides a cartridge which has a gradually narrowing bore with an elliptical cross section.

The present invention, in some embodiments thereof, provides a cartridge which has a gradually narrowing bore with a circular cross section.

The present invention, in some embodiments thereof, provides a cartridge which effects symmetric folding, with a rotational symmetry with respect to a center of a bore, or passageway, in the cartridge.

The present invention, in some embodiments thereof, provides a cartridge which effects symmetric folding, in a “C”-shaped curve, or a “U”-shaped curve.

The present invention, in some embodiments thereof, provides a cartridge which effects “anti-symmetric” folding, in an “S”-shaped curve.

The present invention, in some embodiments thereof, provides a kit which includes an IOL injection cartridge and an IOL. The IOL is optionally paired with the IOL injection cartridge, so that the lens-curling surfaces are suited to optimally curl the paired IOL, taking into account the paired IOL power (diopter) and the paired IOL size (lens diameter and additional haptics size).

The present invention, in some embodiments thereof, provides an IOL injection cartridge which is transparent or semi-transparent, enabling a practitioner to view the IOL within the IOL injection cartridge, to ascertain that the IOL is correctly positioned before folding, and/or to ascertain that the IOL is correctly folded before injecting the IOL within an eye.

The present invention, in some embodiments thereof, provides an IOL injection cartridge which is configured for storing an IOL, optionally in an unfolded configuration, and/or as a sterile kit, optionally for long periods of time, such as hours, days, weeks, months, years—longer than just inserting an IOL into a cartridge moments before insertion into an eye.

The present invention, in some embodiments thereof, provides a kit which includes an IOL injection cartridge and an IOL.

The present invention, in some embodiments thereof, provides a kit in which the IOL is optionally stored within the IOL injection cartridge.

The present invention, in some embodiments thereof, provides a kit in which a slippery agent for reducing friction between the IOL and the cartridge is also stored.

The present invention, in some embodiments thereof, provides a kit which includes an IOL injection cartridge, an IOL, and a compatible IOL injecting device which includes a plunger for pushing the IOL along the IOL injection cartridge.

The present invention, in some embodiments thereof, provides a cartridge with a symmetric bore. The symmetric bore ensures a folding of the IOL from both sides, and reduces the amount of energy imparted to the lenses in the lens-curling, or lens-folding, process. The symmetric bore aids in avoiding damage to the IOL during the lens-curling process. A gradual, smooth, folding of the lens also ensures positioning and travel of IOL haptics down the bore, and aids in avoiding damage of the IOL haptics.

The present invention, in some embodiments thereof, ensures symmetric folding with minimal risk of damage to the IOL haptics.

The present invention, in some embodiments thereof, ensures upward symmetric folding with minimal risk of damage to the IOL haptics.

In some embodiments of the invention, the cartridge 100 is optionally produced of plastic, such as, by way of a non-limiting example, polypropylene and/or low-density polyethylene.

In some embodiments of the invention, the cartridge 100 may be coated, optionally on the inner sides of the passageways, with a slippery agent, at time of manufacture, and/or at time of insertion of an IOL into the cartridge 100, and/or at a time before the lens is pushed through the cartridge, for further reducing friction between the IOL and the cartridge.

In some embodiments of the invention, an angle of passageway walls which gradually fold the IOL are designed to minimize friction.

The present invention, in some embodiments thereof, includes a bore, or passageway, which gradually narrows, effecting a folding of the IOL as the IOL is pushed through the passageway.

In some embodiments, the passageway includes three sections: a first section of the passageway which is large enough to contain an unfolded IOL, a second section of the passageway with angled passageway walls designed to fold the IOL, and a third section of the passageway which has substantially parallel walls, optionally acting as a nozzle, or conduit, to guide a folded IOL into an incision in an eye.

In some embodiments of the invention, a plurality of angles of passageway walls is configured to gradually fold the IOL. In some embodiments of the invention, the plurality of angles of passageway walls are in the second section of the passageway which is described in the paragraph above. When different angles are included in the passageway, the number of different angles may be, by way of a non-limiting example, two, three, four different angles, or more. By way of a non-limiting example, a first angle may be designed to achieve about 20-25% folding of the IOL, a second angle may be designed to achieve approximately an additional 20-25% folding of the IOL, a third angle may be designed to achieve approximately an additional 20-25% folding of the IOL, and a fourth angle may be designed to achieve full folding of the IOL.

In some embodiments of the invention, a cross-sectional area of a final passageway, in which the IOL is fully folded, is equal to a cross sectional of a fully folded IOL.

It is noted that an IOL is typically flexible, and the cross sectional area of the final passageway may be somewhat smaller than the cross sectional area of a fully folded IOL, and the IOL will still squeeze through due to its flexibility.

In some embodiments of the invention, the angles of the passageway walls are configured to change gradually and smoothly along the length of the device.

The present invention, in some embodiments thereof, may be a one part cartridge which includes a section for an unfolded IOL, a section through which the IOL is pushed and which folds the IOL into a compact, folded shape, and a nozzle through which the folded shape is pushed into an eye.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

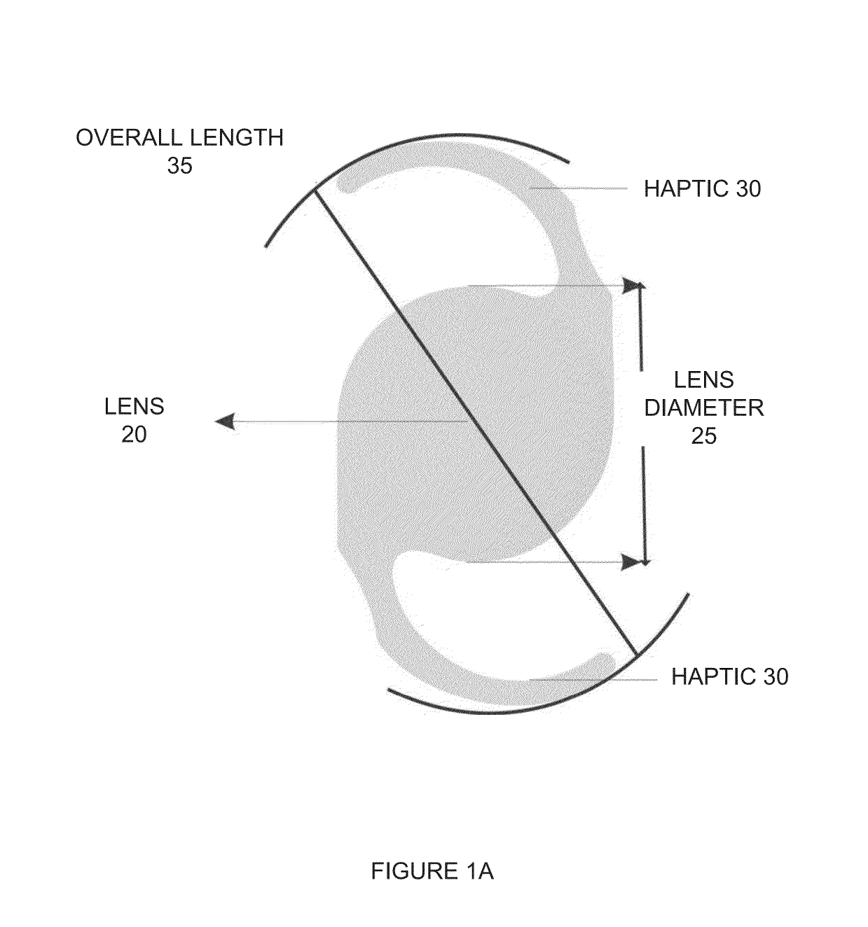

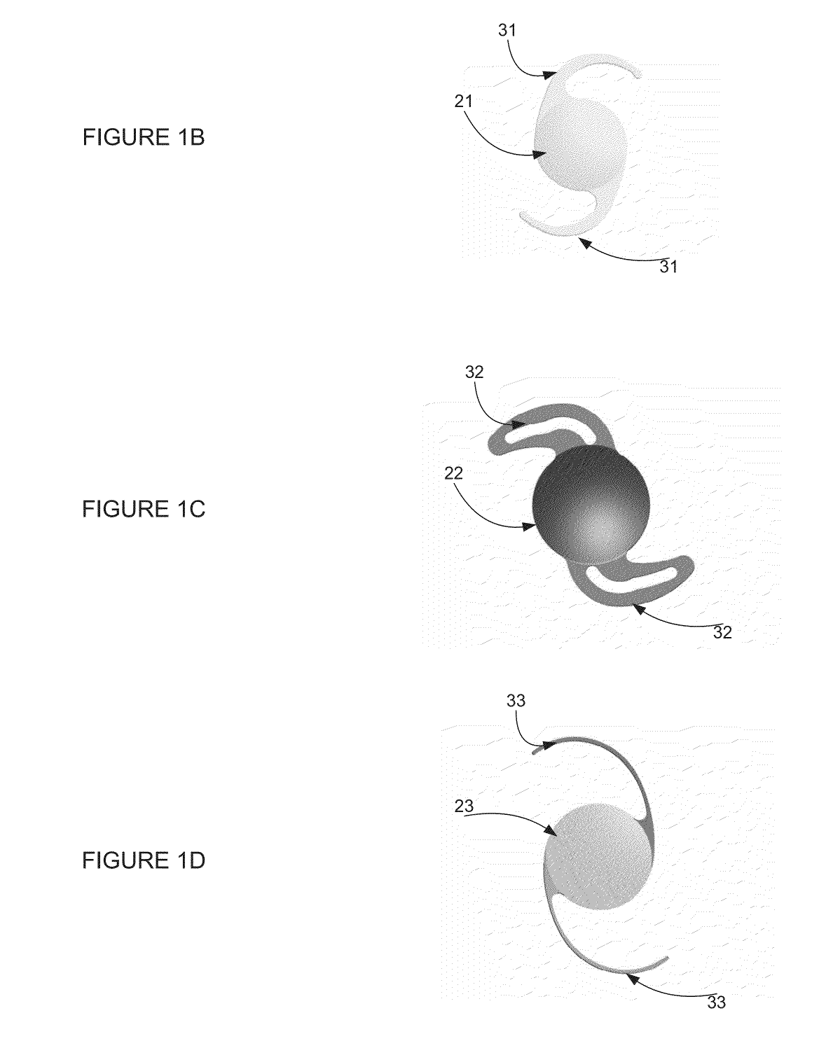

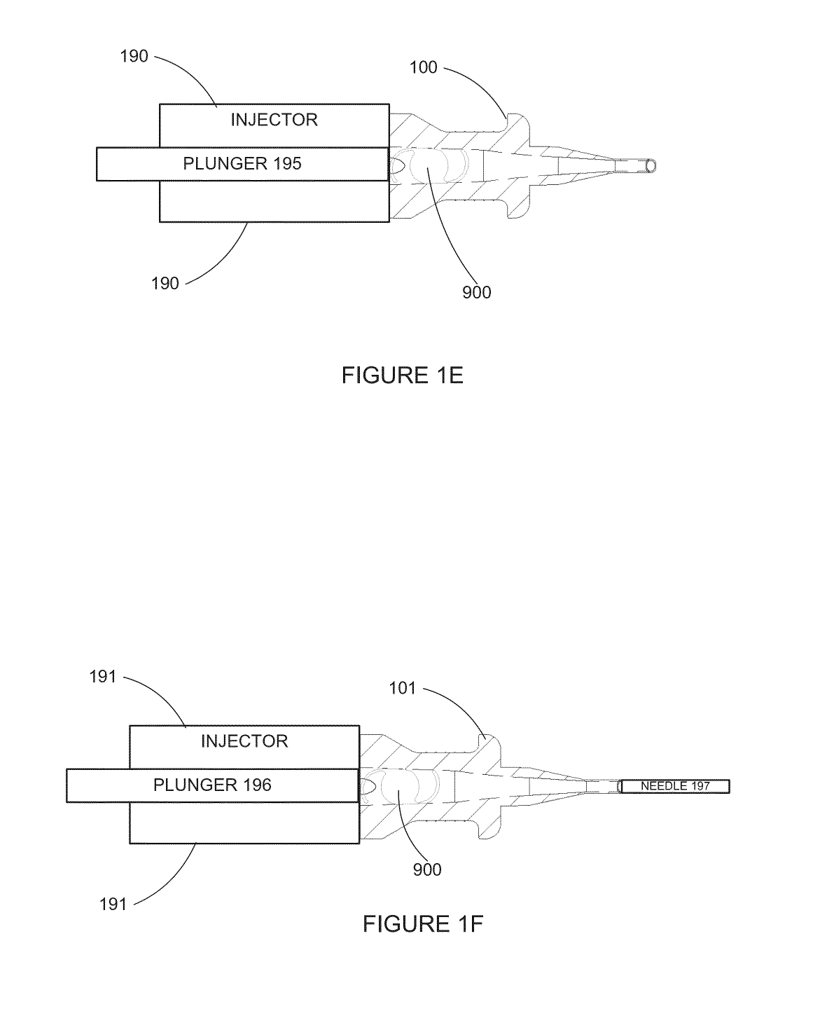

Reference is now made to FIG. 1A, which is a simplified illustration of an example intraocular lens (IOL), showing a lens 20, also termed Optics, and attached haptics 30.

The example IOL of FIG. 1A has a lens diameter 25 of 6.0 millimeters, when not folded. It is noted that typically such lenses have diameters of 5.75 millimeters to 6.25 millimeters, when not folded.

It is noted that embodiments of the invention described herein are suitable for lenses having diameters of a range of at least 5.75 millimeters to 6.25 millimeters; overall length including haptics, of up to 11-13 millimeters; and diopters of the lenses over a range of at least −10 D to +35 D, even though the thickness of the lens 20 (of FIG. 1A) increases as diopter increases.

It is noted that embodiments of the invention described herein are suitable for both hydrophilic and hydrophobic foldable lens materials.

The example IOL of FIG. 1A has attached haptics 30, which increase the not-folded length, at least along one direction, of the IOL to 12.5 millimeters, when the IOL is not folded.

Haptics 30 are optionally used to hold the lens 20 in place in an eye after the lens is inserted into the eye. Haptics typically take a form of a flange which is connected to an edge of the lens 20, and/or a form of generally radially projecting filaments of various configurations (J-shaped, C-Shaped etc.).

It is noted that embodiments of the invention described herein are suitable for foldable IOLs presently in use, including hydrophilic acrylate lenses and hydrophobic acrylate lenses. IOLs made of different materials may have different refractive indexes, for example in a range between 1.4 and 1.6. Thickness of the lenses depends on diopter as well as on refractive index. As diopter increases thickness of the lenses increases, as refractive index decreases thickness of the lenses increases. However, due to flexible nature of the materials making up the foldable IOL, embodiments of the present invention can optionally handle diopters in the range of at least −10 D to +35 D.

IOL designs other than the IOL depicted in FIG. 1A also exist. IOL injection cartridges constructed according to embodiments of the present invention work with different IOL and haptics design. The passageways fold the lens, while maintaining the haptics forward and backward of the lens while the lens is pushed along the passageways.

Reference is now made to FIGS. 1B-1D, which are simplified illustrations of examples of different IOL designs.

FIG. 1B depicts an example single piece IOL, which includes an example lens 21 and example haptics 31, made in one piece.

FIG. 1C depicts a different example IOL, which includes an example lens 22 and example haptics 32, in which the haptics 32 is made in a double haptics design.

FIG. 1C depicts yet another example IOL, which includes an example lens 23 and example haptics 33, in which the two haptics 33 which are depicted are made as separate pieces, connected to the lens 23. The design depicted in FIG. 1C is termed a three piece IOL. In some examples of three piece IOLs, the optic of the lens 23 is made of hydrophilic and/or hydrophobic acrylate, and the haptics 33 are optionally made of a less flexible material. The material of the haptics 33 may be made of polyMethyl MethAcrylate (pMMA), or PolyPropylene (PP), or some other medical grade materials.

Reference is now made to FIGS. 1E and 1F, which are simplified illustrations of an example embodiment of an IOL injection cartridge, functionally connected to an IOL injector.

FIG. 1E depicts a block diagram of an example embodiment of an IOL injection cartridge 100, removably connected to an IOL injector 190. The IOL injector 190 includes a plunger 195, which, during operation of the IOL injector 190, pushes through a passageway in the IOL injection cartridge 100, pushing an IOL 900 through the passageway. The IOL 900 is folded by a gradual narrowing of the passageway, and exits the IOL injection cartridge 100 into an eye.

FIG. 1E depicts a block diagram of an example embodiment of another example embodiment of an IOL injection cartridge 101, removably connected to another example embodiment of an IOL injector 191. The IOL injector 191 includes a plunger 196, which, during operation of the IOL injector 191, pushes through a passageway in the IOL injection cartridge 101, pushing an IOL 900 through the passageway. The IOL 900 is folded by a gradual narrowing of the passageway, and exits the IOL injection cartridge 100 into a needle 197, which may be part of the IOL injector 191, and through the needle 197 into an eye. The term needle is used herein to mean an additional tube-shaped element, which has a passageway through which the IOL 900 can pass.

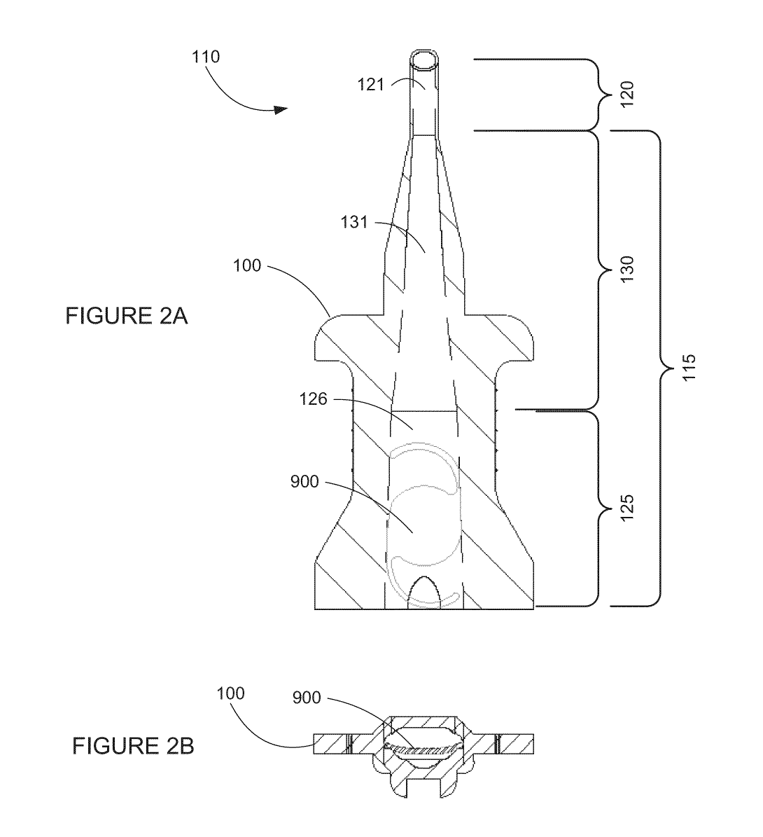

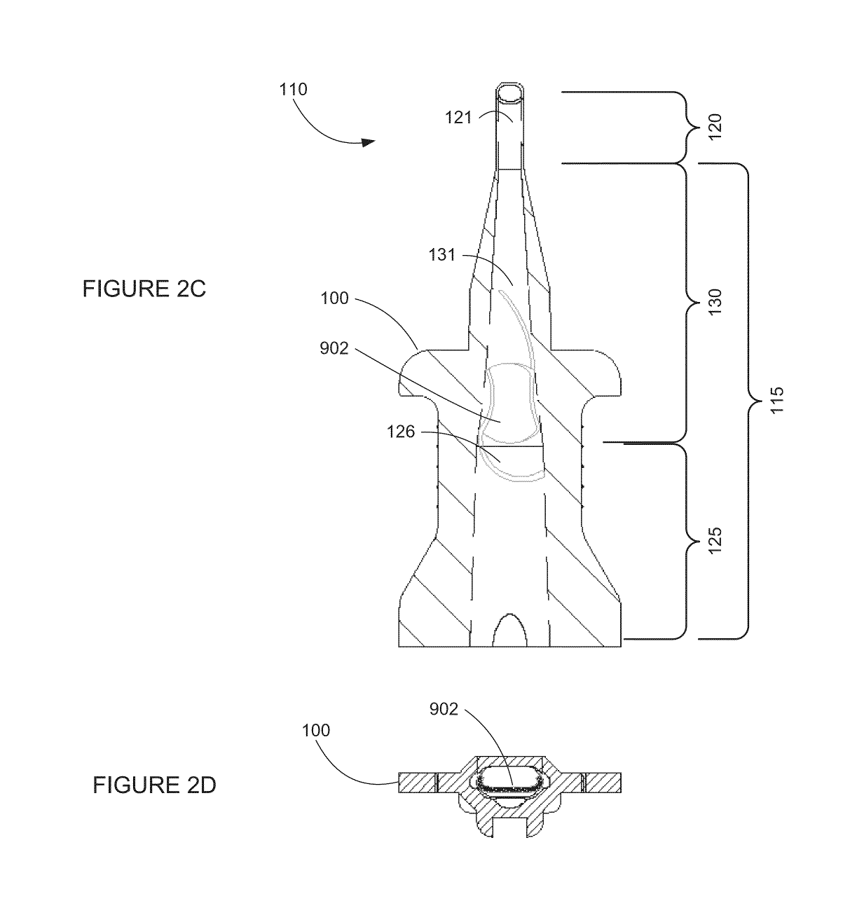

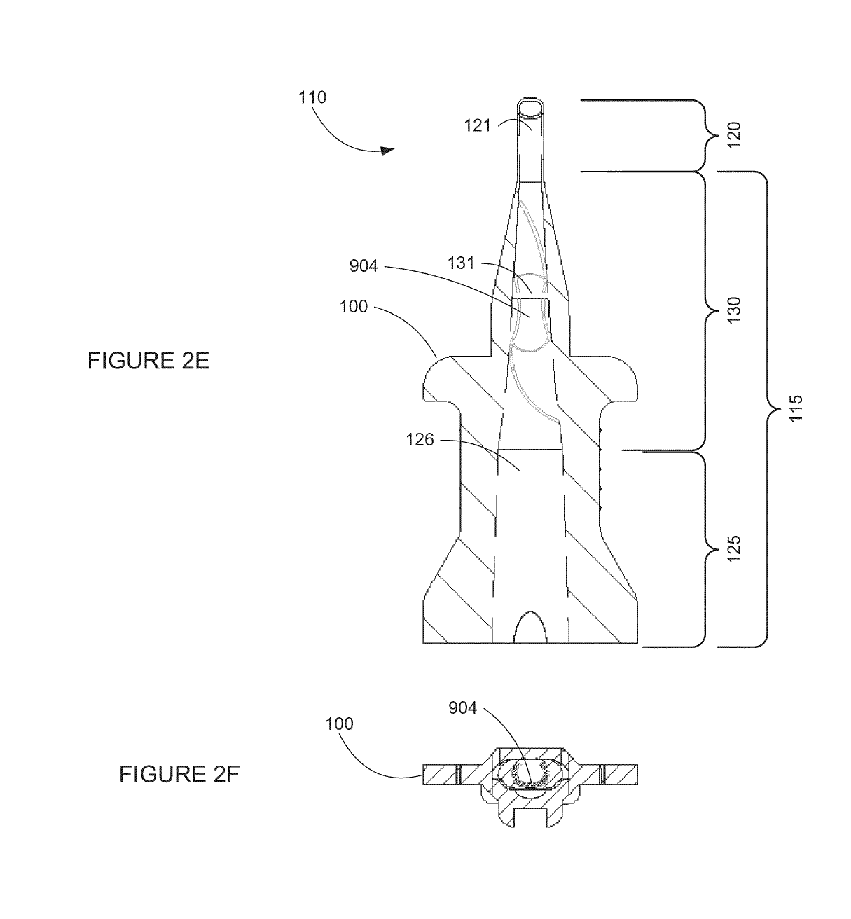

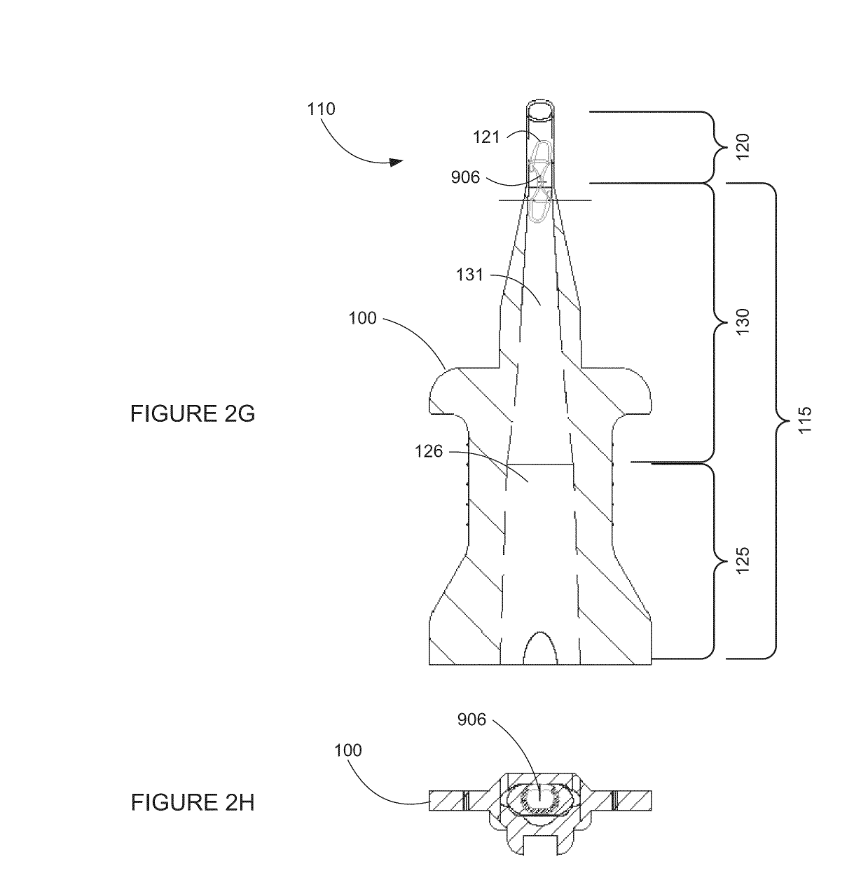

Reference is now made to FIGS. 2A-2H, which are simplified illustrations of an example embodiment of an IOL injection cartridge 100, constructed according to an example embodiment of the invention, and of an IOL advancing along passageways in the cartridge 100;

FIG. 2A depicts a cross section view 110 along a length of the cartridge 100, and FIG. 2B depicts a cross section view across the cartridge 100.

The cartridge 100 includes a body portion 115, and a nozzle portion 120.

The body portion 115 includes a first section 125 which is shaped to include a first lens delivery passageway 126 configured to contain an unfolded IOL 900.

In some embodiments of the invention, the first lens delivery passageway 126 is larger than the IOL 900, and the IOL may optionally be maneuvered within the first lens delivery passageway 126 with an instrument, such as, for example, forceps, to get a correct position of the IOL inside the cartridge 100.

The body portion 115 includes a second section 130, which is shaped to include a second lens delivery passageway 131 connected to the first passageway 126, the second passageway 131 configured to fold the IOL when the IOL is pushed through the second passageway.

The nozzle portion 120 is shaped to include a third lens delivery passageway 121 connected to the second passageway 131, the third passageway 121 configured to transfer the folded IOL into an incision in an eye when the folded IOL is pushed through the nozzle.

The cross section view of FIG. 2B across the cartridge 100 is a cross section view somewhere along the first section 125, and the cross section view also depicts the unfolded IOL 900.

In some embodiments of the invention, the third passageway 121 of the nozzle portion 120 transfers the folded IOL from the cartridge 100 into yet another, fourth passageway (not shown), optionally part of a device which transfers the folded IOL into an incision in an eye when the folded IOL is pushed through the fourth passageway. The fourth passageway may optionally be part of an IOL injector (not shown), into which the cartridge 100 and the IOL are fitted before inserting an IOL into a patient's eye.

It is noted that in some embodiments of the invention the cartridge 100 may be curved. In some embodiments, the curve is along the entire length of the cartridge 100. In some embodiments, the curve is only along the nozzle portion 120 of the cartridge 100.

FIG. 2C depicts the cross section view 110 along the length of the cartridge 100, and FIG. 2D depicts another cross section view across the cartridge 100.

The cross section view of FIG. 2D across the cartridge 100 is a cross section view somewhere along the second section 130, and the cross section view of FIG. 2D also depicts a partially folded IOL 902, which is a depiction of the unfolded IOL 900 after it has been pushed along the first passageway 126 and at least partially along the second passageway 131.

The cross section view of FIG. 2D across the cartridge 100 depicts an example of a round-cornered rectangular cross section.

It is noted that a slope of the second passageway 131 is configured to symmetrically fold the IOL 900. The symmetrical folding optionally distributes stress on the IOL 900 while folding, minimizing possible damage to the IOL and to haptics connected to the IOL.

In some embodiments of the invention, a plunger pushing the IOL along the passageways of the cartridge 100 touches only the lens 20 (of FIG. 1A) and not the haptics 30.

FIG. 2E depicts the cross section view 110 along the length of the cartridge 100, and FIG. 2F depicts a cross section view across the cartridge 100.

The cross section view of FIG. 2F across the cartridge 100 is a cross section view somewhere along the second section 130, further toward the nozzle portion 120 than the cross section view of FIG. 2D, and the cross section view of FIG. 2F also depicts a more folded IOL 904 than the partially folded IOL 902 depicted in FIG. 2D.

FIG. 2G depicts the cross section view 110 along the length of the cartridge 100, and FIG. 2H depicts a cross section view across the cartridge 100.

The cross section view of FIG. 2H across the cartridge 100 is a cross section view somewhere along the nozzle portion 120, and the cross section view of FIG. 2H depicts an even more folded IOL 906 than the partially folded IOL 904 depicted in FIG. 2F.

It is noted that first lens delivery passageway 126, the second passageway 131, and the third passageway 121 may be viewed as one passageway, optionally having walls at different slopes, for containing an IOL, folding the IOL, and inserting the IOL into an eye.

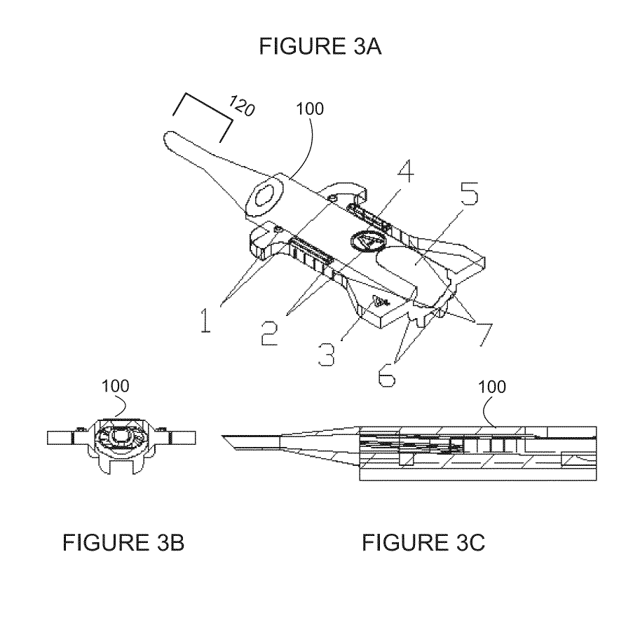

Reference is now made to FIGS. 3A-3C, which are additional simplified illustrations of the example embodiment of the cartridge 100 of FIGS. 2A-2H.

FIGS. 3A-3C depicts additional, optional features of the cartridge 100 of FIGS. 2A-2H, which may assist is making the cartridge especially useful for certain applications.

FIG. 3A is a simplified isometric illustration of the cartridge 100.

FIG. 3B is a simplified end-on illustration of the cartridge 100.

FIG. 3C is a simplified cross sectional illustration of the cartridge 100, cut along a length of the cartridge 100.

FIG. 3A depicts optional studs (1) for fitting the cartridge 100 in an IOL injector. The studs (1) avoid possibility of displacement during the injection of the IOL, especially when forces are applied while pushing the IOL toward the nozzle portion 120 of the cartridge. The studs (1) keep the cartridge 100 cartridge steady in an IOL injector (not shown), and enable proper alignment of the cartridge 100 to the IOL injector (not shown).

FIG. 3A also depicts optional ribs (2) for aiding a correct direction of loading the cartridge 100 in the IOL injector, aiding to avoid human error.

FIG. 3A also depicts an optional identification number (3), which aids traceability of the cartridge 100 in case of any issue with the cartridge 100. The optional identification number may include one or more of: an identification number; a manufacturing lot number; a number identifying in which mold the cartridge 100 was molded—especially useful if multiple cartridges 100 are produced in a multi-cavity mold; a number identifying a diopter of an IOL included in a kit with the cartridge 100; a number identifying a diameter of an IOL included in a kit with the cartridge 100; and a number identifying a model of a compatible IOL injector.

It is noted that the cartridge, in some embodiment thereof, is designed for production using a mold.

It is noted that the cartridge, in some embodiment thereof, is designed for production using injection molding.

In some embodiments, the cartridge is designed for production of multiple cartridges in a multi-cavity mold.

FIG. 3A also depicts an optional marking (4), such as, by way of a non-limiting example, a marking of a capital “A”, for aiding upward (Anterior) loading of the cartridge 100 in the IOL injector, and for assisting in preventing human error while loading the cartridge 100 in the injector.

FIG. 3A also depicts an optional pocket (5), optionally on an anterior side of the cartridge, for ensuring an ideal position of the IOL in the cartridge. The optional pocket (5) makes the placement of IOLs, when the IOLs are not supplied within the cartridge 100, easy and predictable, by allowing an IOL to be placed into the cartridge in an unfolded, unstressed, form.

FIG. 3A also depicts an optional shaping (6) of a posterior side of the cartridge 100, for ensuring a correct mating, position and alignment, of the cartridge 100 in the IOL injector.

FIG. 3A also depicts optional ribs (7), optionally on both sides of the cartridge 100, for assisting in unloading the cartridge 100 from the IOL injector.

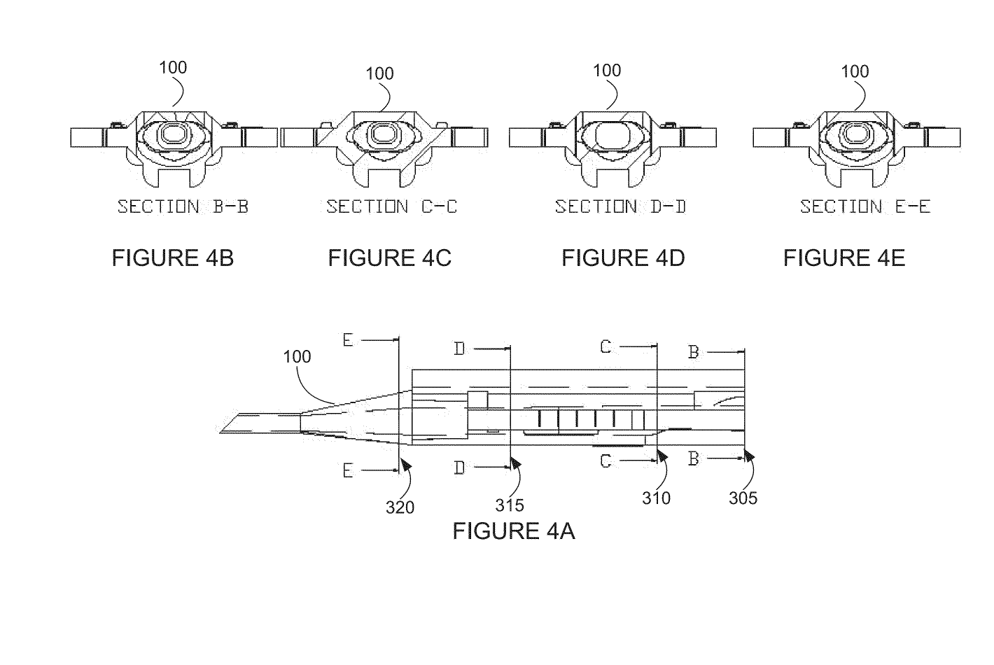

Reference is now made to FIGS. 4A-4E, which are additional simplified illustrations of the example embodiment of the cartridge of FIGS. 2A-2H.

FIG. 4A depicts a simplified side view illustration of the cartridge 100, and four cross-sectional planes B-B 305, C-C 310, D-D 315, and E-E 320.

FIG. 4B depicts a simplified cross sectional illustration of the cartridge 100 along the plane B-B 305.

FIG. 4C depicts a simplified cross sectional illustration of the cartridge 100 along the plane C-C 310.

FIG. 4D depicts a simplified cross sectional illustration of the cartridge 100 along the plane D-D 315.

FIG. 4E depicts a simplified cross sectional illustration of the cartridge 100 along the plane E-E 320.

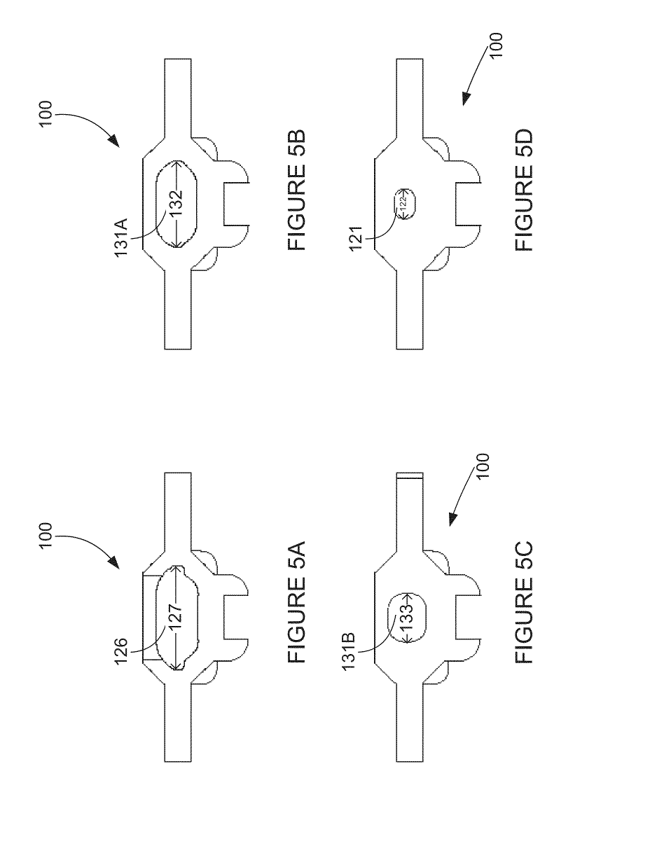

Reference is now made to FIGS. 5A-5D, which are additional simplified illustrations of the example embodiment of the cartridge of FIGS. 2A-2H.

An example embodiment of the cartridge 100 optionally has four sections, depicted in FIGS. 5A-5D, with passageways for an IOL narrowing from start to end. The passageways effect folding, optionally upward folding, of the lens, while pushing the lens forward from start to end.

FIG. 5A depicts a simplified cross sectional illustration of the cartridge 100 somewhere along the first section 125 of the cartridge 100 of FIG. 2A, showing a cross section of the first lens delivery passageway 126. The cross section of the first lens delivery passageway 126 is shaped and sized to ensure proper holding of an IOL with minimal contact of the lens body with an inner surface of the cartridge 100, to avoid any structural damage to the lens and/or to the haptics. In some embodiments of the invention, a diameter of the first lens delivery passageway 126 is substantially equal to a diameter of an IOL (typically 5.75 millimeters to 6.25 millimeters) plus haptics attached to the IOL.

In an example embodiment of the cartridge 100, the width 127 of the first lens delivery passageway 126 is approximately 6.0 millimeters, sized to hold an unfolded IOL.

FIG. 5B depicts a simplified cross sectional illustration of the cartridge 100 somewhere along the second section 130 of the cartridge 100 of FIG. 2A, showing a first cross section 131A of the second lens delivery passageway 131. The first cross section 131A of the second lens delivery passageway 131 is shaped and sized to effect a symmetric folding of the IOL, and to enable smooth traveling of the lens inside the cartridge. Symmetric folding of the IOL may optionally be either upward or downward, relative to a plane defined by a circumference of the lens.

In an example embodiment of the cartridge 100, the width 132 of the first cross section 131A is approximately 4.8 millimeters, sized to fold the IOL mildly, optionally up to 20%-25% of full folding, while traveling from the first section 125 to a location corresponding to the first cross section 131A of the second lens delivery passageway 131.

FIG. 5C depicts a simplified cross sectional illustration of the cartridge 100 somewhere along the second section 130 of the cartridge 100 of FIG. 2A, showing a second cross section 131B of the second lens delivery passageway 131. The second cross section 131B is further along the second lens delivery passageway 131, toward the nozzle portion 120 of the cartridge 100 than is the first cross section 131A. The second cross section 131B of the second lens delivery passageway 131 is shaped and sized to effect yet more folding of the IOL, for example to effect 75% of the entire desired folding, in terms of reduction of cross-sectional area of the folded IOL, in order to prepare the IOL for passing through the nozzle portion 120 and out of the cartridge 100. The shape and size of the second cross section 131B also enable smooth travel of the IOL, with a gradual folding of the lens, and optionally minimizes the amount of energy imparted to the lens by the folding.

In an example embodiment of the cartridge 100, the width 133 of the second cross section 131B is approximately 2.8 millimeters, sized and shaped to fold the IOL symmetrically while traveling from the location corresponding to the first cross section 131A of the second lens delivery passageway 131 to the location corresponding to the second cross section 131B of the second lens delivery passageway 131.

FIG. 5D depicts a simplified cross sectional illustration of the cartridge 100 somewhere along the nozzle portion 120 of the cartridge 100 of FIG. 2A, showing a cross section of the third lens delivery passageway 121. The cross section of the third lens delivery passageway 121 is shaped and sized to ensure symmetric and 100% folding of the lens when passing the lens body from the cartridge 100 and to enable smooth travel of the IOL without damage to the haptics.

In an example embodiment of the cartridge 100, the width 122 of the third lens delivery passageway 121 of the nozzle portion 120 is approximately 1.7 millimeters, sized and shaped to achieve maximal desired folding of the IOL while traveling through the nozzle portion 120. In some embodiments of the invention the width 122 of the third lens delivery passageway 121 of the nozzle portion 120 may be in a range between 1.75 millimeters and 3.25 millimeters.

It is noted that the dimensions provided above with reference to the example embodiment of FIGS. 5A-5D should be referred to with a tolerance of +/−0.2 millimeters.

A cross sectional area of the passageway 121 upon exiting the nozzle section 120 optionally varies from 1.75 square millimeters to 3.25 square millimeters for different incision sizes, optionally according to a surgeon's choice, and/or for different lens diopters, and/or for different lens diameters, optionally according to an IOL selected by a surgeon and/or a medical practitioner.

In some embodiments of the invention a cross sectional area of the passageway 121 upon exiting the nozzle section 120 optionally substantially equals an area of a fully folded IOL plus haptics. Such an area can be determined by fully folding, outside of the cartridge 100, a sample IOL of the diopter and diameter which is to be used within the cartridge 100.

In some embodiments of the invention, angles of the narrowing passageways are optionally variable between locations along the passageways, but are equal on both sides of a passageway, to effect symmetric folding of the lens.

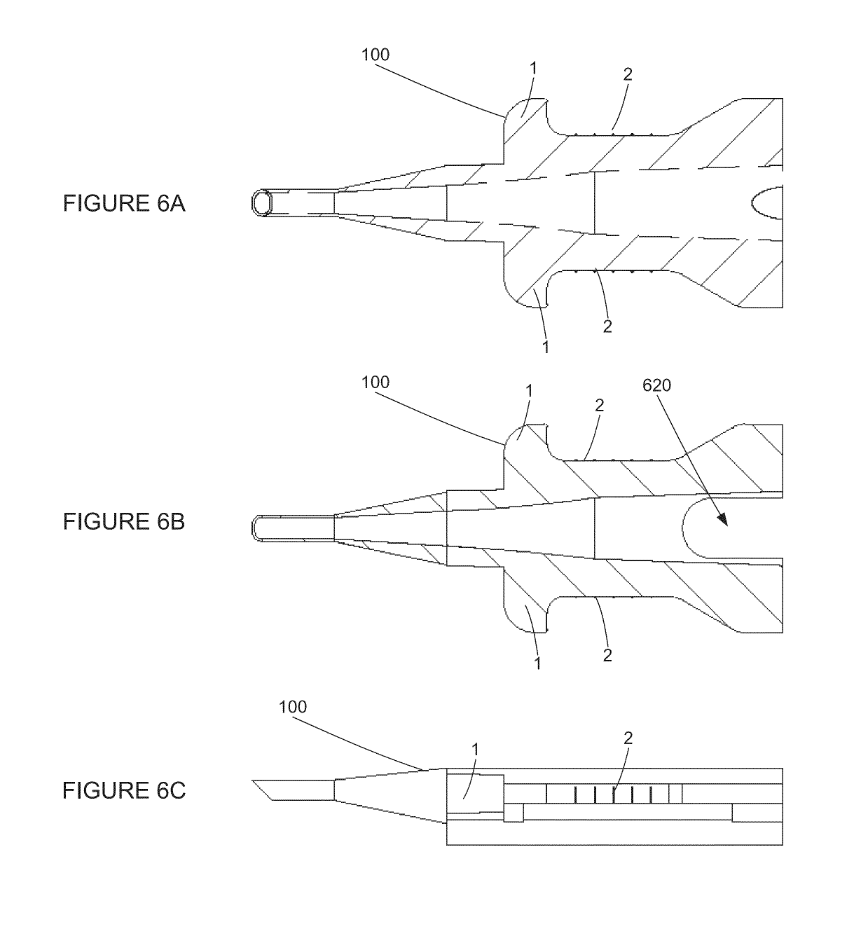

Reference is now made to FIGS. 6A-6C, which are additional simplified illustrations of the example embodiment of the cartridge of FIGS. 2A-2H.

FIG. 6A depicts a simplified illustration of a Posterior side of the cartridge 100, and includes the optional studs (1), and the optional ribs (2) of FIG. 3A.

FIG. 6B depicts a simplified illustration of an anterior side of the cartridge 100, including the optional studs (1) of FIG. 3A, the optional ribs (2) of FIG. 3A, and including a recess 620 for inserting an IOL (not shown) into the cartridge 100.

FIG. 6C depicts a simplified side view illustration of the cartridge 100, including the optional studs (1), and the optional ribs (2) of FIG. 3A.

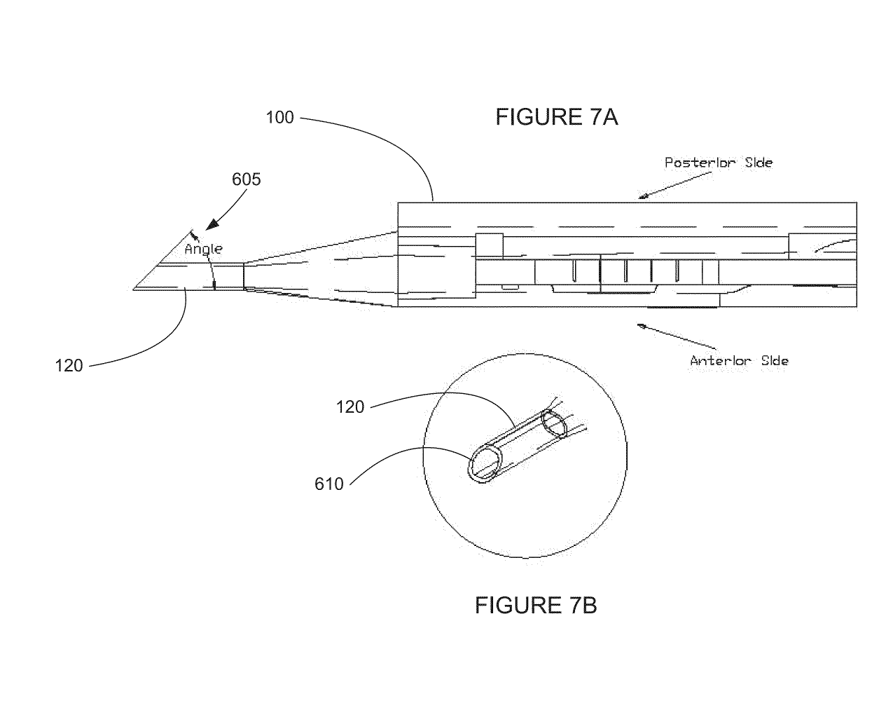

Reference is now made to FIGS. 7A and 7B, which are additional simplified illustrations of the example embodiment of the cartridge 100 of FIGS. 2A-2H.

FIG. 7A depicts a simplified side view of the cartridge 100, and an optional angle 605 at an end of the nozzle portion 120 of the cartridge 100.

In some embodiments of the invention, the angle 605 may have a value anywhere between 15 and 60 degrees, and even up to 85 degrees.

Values of cartridge tip angle from 15 to 60 degrees effect smooth delivery of the lens through the cartridge 100, and/or reduce an amount of energy imparted to the lens, and/or reduce a potential for damage to the lens.

FIG. 7B depicts an isometric, magnified, view of a portion of the cartridge 100, depicting the nozzle portion 120 and a tip 610 of the nozzle portion 120, cut at the angle 605 of FIG. 7A.

In some embodiments of the invention, where the cartridge 100 serves for inserting an IOL into an incision in an eye, the angle 605 is an angle designed to be inserted into the incision. An angled tip of the nozzle portion 120 may be easily inserted into an incision in an eye, much as an angled tip of a syringe needle is inserted with less effort. Additionally, a folded IOL starts unfolding, gradually, while being pushed out of an angled tip of the nozzle portion 120.

In some embodiments of the invention, where the cartridge 100 is inserted into an IOL injector (not shown), and it is the IOL injector and not the cartridge 100 which serves for inserting the IOL into an incision in an eye, the angle 605 is an angle designed to ensure placement of the cartridge 100 so that the third passageway 121 of the nozzle portion 120 abuts a fourth passageway present within the injector. In such embodiments the injection device uses the cartridge to feed a folded IOL into a tube or nozzle built into the injection device.

It is expected that during the life of a patent maturing from this application many relevant intraocular lenses (IOLs) will be developed and the scope of the term IOL is intended to include all such new technologies a priori.

As used herein the term “about” refers to ±10%.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A cartridge for implantation of a deformable intraocular lens (IOL) through a small incision in the eye, comprising:
a body portion comprising:
a first section comprising a first lens delivery passageway configured to contain an IOL readied for implantation; and
a second section comprising a second lens delivery passageway connected to the first passageway, configured to fold the IOL when the IOL is pushed through the second passageway, in which said second lens delivery passageway comprises a symmetric, gradually narrowing bore whose walls narrow at a plurality of angles comprising at least three angles, designed for minimizing stress on the IOL when it folds; and
a nozzle portion extending from said body portion, said nozzle portion comprising a third passageway and a tip for insertion through the incision in the eye, the second passageway extending to the third passageway, the third passageway configured to transfer the folded IOL into an incision in an eye when the folded IOL is pushed through the nozzle.

2. The device according to claim 1, in which the first lens delivery passageway is configured to contain an IOL in which the lens part is not folded.

3. The device according to claim 1, in which the second lens delivery passageway comprises passageway walls which progressively narrow the second passageway, the narrowing of the walls being symmetrical around a long axis connecting the first, second, and third passageway.

4. The device according to claim 1 in which said second lens delivery passageway comprises walls designed to effect anti-symmetric folding of said IOL into an S-shaped curve.

5. The device according to claim 1 in which passageway walls progressively narrow at angles which change along the passageways.

6. The device according to claim 1 in which passageway walls progressively narrow at an angle configured for minimizing stress on the IOL based, at least in part, on a diameter of the IOL.

7. The device according to claim 1 in which passageway walls progressively narrow at an angle configured for minimizing stress on the IOL based, at least in part, on a diopter of the IOL.

8. The device according to claim 1, having studs for connection to an IOL injector which includes a plunger for pushing the IOL along the lens delivery passageways.

9. The device according to claim 1, having a shape for mating to an IOL injector which includes a plunger for pushing the IOL along the lens delivery passageways.

10. A kit for implantation of a deformable intraocular lens (IOL) through a small incision in the eye, comprising the cartridge of claim 1 and an unfolded intraocular lens.

11. The kit according to claim 10 in which the unfolded intraocular lens is stored within the first lens delivery passageway.

12. The kit according to claim 10 and further comprising a slippery agent for reducing friction between the IOL and the cartridge.

13. A method for manufacture of a cartridge for implantation of a deformable intraocular lens (IOL) through a small incision in the eye,
(a) the cartridge comprising:
a body portion comprising:
a first section comprising a first lens delivery passageway configured to contain an unfolded IOL readied for implantation; and
a second section comprising a second lens delivery passageway connected to the first passageway, configured to fold the IOL when the IOL is pushed through the second passageway, in which said second lens delivery passageway comprises a symmetric, gradually narrowing bore whose walls narrow at a plurality of angles comprising at least three angles, designed for minimizing stress on the IOL when it folds; and
a nozzle portion connected to said body portion, said nozzle portion comprising a third passageway and a tip for insertion through the incision in the eye, the second passageway extending to the third passageway, the third passageway configured to transfer the folded IOL into an incision in an eye when the folded IOL is pushed through the nozzle; and
(b) the manufacturing method comprising forming the cartridge by molding material to produce the cartridge.

14. The method according to claim 13, in which the molding comprises injection molding.

15. The method according to claim 13 in which the material comprises a plastic.

16. The method according to claim 13 in which the material comprises one of the group consisting of:
polypropylene; and
low-density polyethylene.

17. The kit according to claim 11 and further comprising a slippery agent for reducing friction between the IOL and the cartridge.
